(19) United States
(12) Patent Application Publication
(10) Pub. No.: US 2023/0120489 A1
(43) Pub. Date: Apr. 20, 2023

(54) PERFUSION MODULE AND PERFUSION CULTURE SYSTEM

(71) Applicant: Yukuan SHANG, Langfang, Hebei (CN)

(72) Inventor: YUKUAN SHANG, LANGFANG, HEBEI PROVINCE (CN)

(21) Appl. No.: 17/908,513

(22) PCT Filed: Apr. 29, 2020
(86) PCT No.: PCT/CN2020/087684

§ 371 Date: Aug. 31, 2022

(30) Foreign Application Priority Data
Mar. 6, 2020 (CN) 202010152065.3
Mar. 6, 2020 (CN) 202020271369.7

(51) Int. Cl. C12M 1/00

(52) U.S. Cl. CPC C12M 29/10; C12M 29/08; C12M 29/04; C12M 23/40

(57) ABSTRACT

A perfusion module and a perfusion culture system are provided, including: a bioreaction module, including at least one bioreactor; a perfusion module, including at least one perfusion chamber, each perfusion chamber is connected to a pressure regulating pipe for inflow or outflow of an airflow; when there are multiple perfusion chambers, every two adjacent perfusion chambers are connected to each other; at least one bioreactor, the perfusion module, the filtration module are communicated in sequence, if there are more than two perfusion chambers, all of perfusion chambers are set side by side; a cell culture fluid in at least one bioreactor flows into at least one perfusion chamber, flows out from at least one perfusion chamber into at least one filter, and a permeate from at least one filter flows into a harvesting device. The present disclosure avoids high shear forces in the perfusion culture system.

### TECHNICAL FIELD

The present disclosure relates to the field of perfusion culture devices, and in particular, to a perfusion module and a perfusion culture system.

### BACKGROUND

Among current large-scale fermentation processes of animal cells, the stirred suspension continuous fermentation process is the mainstream, especially for large-scale production of recombinant proteins such as antibodies. In industrial production, the scaling-up principle and process control of parameters of a suspension culture process are easier to understand and master than those of other fermentation systems, so the suspension culture process is widely adopted in large-scale animal cell fermentation production. The suspension culture process is a fermentation method in which cells are freely suspended in a culture liquid for growth and proliferation. This technology has been developing rapidly and is reaching maturity.

A perfusion culture process is a common fermentation process, which mostly adopts a stirred cell fermentation system, or a pipe system. During the fermentation process, the culture liquid continuously flows into a fermenter at a certain flow rate, and then flows out of the fermenter at the same flow rate. One advantage of the perfusion culture process is that cell debris and by-products can be continuously removed during the fermentation process, to reduce possible negative effects of various enzymes released after cell death on the product. At the same time, a constant culture environment is provided because fresh culture liquid is infused continuously. For the above reasons, a higher yield per volume can be obtained. High-density fermentation of cells can be achieved because most perfusion systems adopt cell interception and recovery systems.

Currently, the choice of animal cell culture methods is determined by the characteristics of cell growth and the nature of the target protein. One significant advantage of the perfusion culture process is that the proteins can be isolated in time along with the culture medium, and a short residence time of the proteins in the reactor makes them less susceptible to degradation by various hydrolytic enzymes in the culture system, which improves the quality of proteins. This feature is extremely advantageous for the production of chemically unstable proteins, such as enzymes, coagulation factors and the like. In contrast, when cell growth and the quality of proteins are not limited by the culture method, for example, for the production of drugs such as antibodies that are relatively chemically stable, the choice between cell perfusion culture and fed-batch culture needs to be made after a holistic assessment that takes into consideration cost, benefit, scale, risk, and operation flexibility.

Biotechnology companies are increasingly inclined to develop highly flexible and efficient manufacturing processes that are compatible with diverse manufacturing conditions. The perfusion culture process has been widely used in the bioengineering industry as an effective means of achieving increased yields of low-stability recombinant proteins. The amount of proteins produced by the perfusion culture process with small bioreactors is comparable with the amount of proteins produced by large-scale production in the fed-batch culture process, thereby enabling miniaturization of culture scale and increasing operation flexibility.

With the large-scale application of suspension cells, the suspension cell perfusion culture process is also increasingly developed. Cell interception is one step of the suspension cell perfusion culture process, and how to effectively intercept cells without causing damage to them has become the focus and challenge of the suspension cell perfusion culture process. Current cell retention devices are mainly designed based filtration, sedimentation, and centrifugation, and include rotary filters, vortex filters, tilting settlers, spin separators, and the like. Filtration-based cell retention devices can isolate cells completely (the isolation rate is 100%), but their filter membranes are easily clogged by cell debris, defoamers, etc., which eventually leads to the termination of the perfusion culture. Centrifugation of cells for isolation may cause cell damage. Neither sedimentation nor centrifugation can completely intercept cells, and their cell isolation effects are also affected by perfusion rates. In addition, how to scale up easily and effectively is a challenge facing most cell intercepting devices.

Alternating tangential flow (ATF) systems, designed based on hollow fiber interception, are better cell intercepting devices because they have many advantages, such as the ability to effectively relieve membrane clogging, a low shear force. In an ATF system, hollow fibers are connected to one end of a reactor through a pipe, and for the other end of the reactor, a silica gel diaphragm pump is alternately driven by a high-pressure provided by an air pump and a negative pressure provided by a vacuum pump, so that animal cell culture fluid enters and leaves the reactor through the hollow fibers. This process also achieves sufficient flushing of the hollow fibers under low shear forces, and effectively reduces clogging caused by filter cakes accumulated on the hollow fibers. By adopting ATF systems, high cell densities and high protein yields can be achieved at a high perfusion rate, and the ratio of the culture volume to the membrane area of hollow fibers can also be effectively scaled up to mass production.

However, the above-mentioned ATF systems also have the following drawbacks: the largest model of ATF perfusion systems (e.g., ATF10) currently has a processing capacity of 500-800 L/set (or each batch). For reactors of tonnage or larger, multiple ATF systems connected in parallel will be needed, which is costly. In addition, each ATF system has a certain dead volume due to intrinsic design defects, and the reciprocal movement of liquid in the dead volume is detrimental to the cell culture.

Therefore, there is a need fora low-cost, large-scale (i.e., 1000-30,000 L) perfusion culture system.

### SUMMARY

The present disclosure provides a perfusion module, the perfusion module includes at least one perfusion chamber, each perfusion chamber is connected to a pressure regulating pipe for inflow or outflow of an airflow, and the pressure regulating pipe is provided with a pump that enables the pressure regulating pipe to be opened and closed and changes a delivery direction of the airflow; when there are a plurality of perfusion chambers, every two adjacent perfusion chambers are communicated with each other through the pressure regulating pipe.

The present disclosure also provides a perfusion culture system. The perfusion culture system includes a bioreaction module, a perfusion module, and a filtration module.

the bioreaction module includes at least one bioreactor;

a perfusion module includes at least one perfusion chamber, each perfusion chamber is connected to a pressure regulating pipe for inflow or outflow of an airflow, and the pressure regulating pipe is provided with a pump that enables the pressure regulating pipe to be opened and closed and changes a delivery direction of the airflow; when there are a plurality of perfusion chambers, every two adjacent perfusion chambers are connected to each other through the pressure regulating pipe;

a filtration module includes at least one filter, each filter has an inlet end and an outlet end opposite to the inlet end, the inlet end is provided with a cell culture fluid inlet, and the outlet end is provided with a permeate outlet and a cell culture fluid outlet;

the at least one bioreactor, the perfusion module, and the filtration module are communicated in sequence, and if there are more than two perfusion chambers in the perfusion module, all of the perfusion chambers are set side by side; wherein a cell culture fluid in the at least one bioreactor flows into the at least one perfusion chamber in the perfusion module, and then flows out from the at least one perfusion chamber into the at least one filter for filtration, and a permeate from the at least one filter flows into a harvesting device through the permeate outlet.

In an embodiment, the inlet end of each filter is further provided with a medium inlet, and the medium inlet is connected to a medium supply system.

In an embodiment, the cell culture fluid outlet of each filter is connected to the at least one bioreactor through a return pipe, and the cell culture fluid filtered by each filter is returned to the at least one bioreactor through the return pipe.

In an embodiment, the medium supply system is connected to the at least one bioreactor through a medium delivery pipe.

In an embodiment, when the perfusion module includes only one perfusion chamber, a top of the perfusion chamber is communicated with a top of the at least one bioreactor through the pressure regulating pipe.

In an embodiment, the pump is a diaphragm pump.

In an embodiment, each perfusion chamber is connected to an airflow mass flow controller through an airflow delivery pipe.

In an embodiment, the airflow delivery pipe is provided with a gas filter or/and a switching valve.

In an embodiment, the at least one bioreactor is connected to the at least one perfusion chamber through an infusion pipe, and the infusion pipe is provided with an on-off valve to enable the infusion pipe to be opened and closed.

In an embodiment, the filtration module is provided with two filters set side by side, and the two filters work alternately.

As described above, the perfusion culture system provided by the present disclosure have the following beneficial effects: at least one bioreactor, at least one perfusion chamber, and at least one filter are communicated through pipelines and multiple perfusion chamber are provided, to achieve large-scale (i.e., 1000-30,000L) perfusion culture. All of the at least one perfusion chamber is connected to a pressure regulating pipe, so that the airflow flowed out from the perfusion chambers or flowed in the perfusion chambers can be adjusted, achieving positive pressure control of the perfusion chamber, facilitating the flow of the cell culture fluid, and avoiding high shear forces in the perfusion culture system.

### BRIEF DESCRIPTION OF THE DRAWINGS

FIG. 1 is a schematic diagram of a perfusion culture system according to an embodiment of the present disclosure.

FIG. 2 is a structural diagram of a perfusion culture system according to an embodiment of the present disclosure.

### REFERENCE NUMERALS

1 Bioreactor

2, 2a, 2b Perfusion chamber

3, 3a, 3b Filter

4 Medium supply system

5 Harvesting device

6 Mass flow controller

7 Harvesting pump

9 Delivery pump

10 Electromagnetic flow meter

8, 12, 13, 17, 18, 24, 23, 22 Valve

14 Switching Valvemeter

15 Gas filter

16 Pump

11, 20 On-off valve

19 Normally-open valve

21 Protection valve

101 Infusion pipe

102 Return pipe

103 Medium delivery pipe

104 Delivery pipe

105 Pressure regulating pipe

### DETAILED DESCRIPTION

The implementations of the present disclosure are described below through specific examples. Those skilled in the art can easily understand the other advantages and effects of the present disclosure from the content disclosed in this specification.

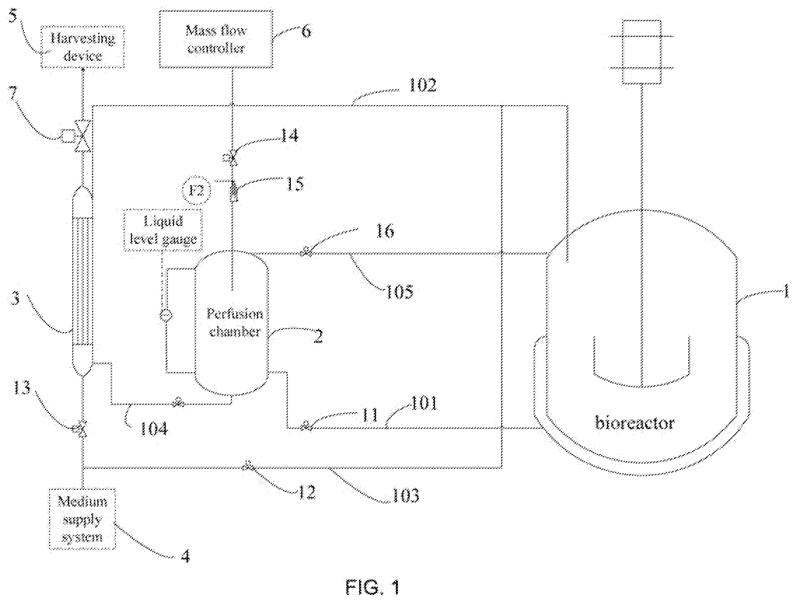

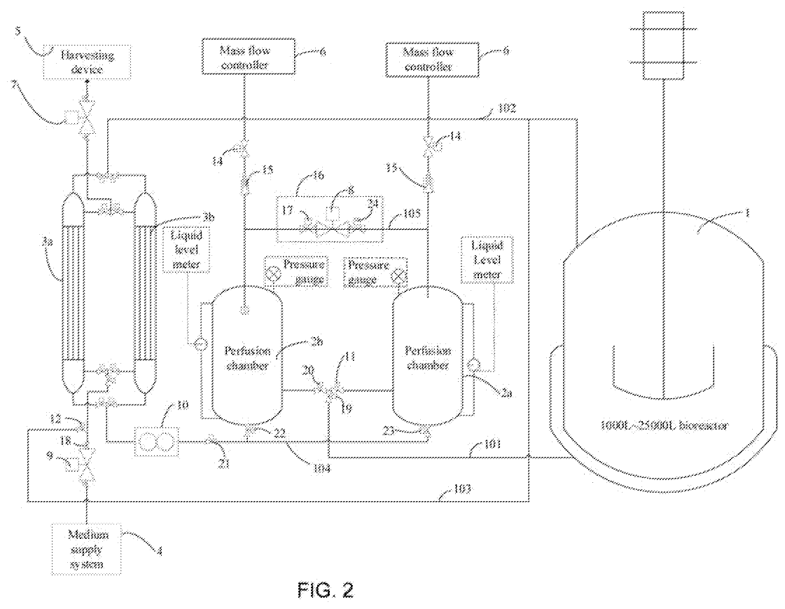

Refer to FIG. 1 and FIG. 2. It should be noted that the structure, proportion, size, etc. illustrated in the drawings of this specification are only used to match the contents disclosed in the specification for the understanding and reading of those skilled in the art, and are not used to limit the conditions under which the present disclosure can be implemented. Any modification of the structure, change of proportion or adjustment of the size, without affecting the efficacy of the present disclosure and the purpose it can achieve, should still fall within the scope of the technical contents disclosed in the present disclosure. At the same time, the terms such as “up”, “down”, “left”, “right”, “middle” and “one” used in this specification are only for the clarity of the description, and are not intended to limit the scope of the present disclosure, and any change or adjustment of relative relationships thereof without substantial change of the technical content herein shall still fall within the scope of the present disclosure.

The present disclosure provides a perfusion module for regulating positive pressure, as shown in FIG. 1 and FIG. 2. The perfusion module includes at least one perfusion chamber 2. Each perfusion chamber 2 is connected to a pressure regulating pipe 105 for inflow or outflow of an airflow, and the pressure regulating pipe 105 is provided with a pump that enables the pressure regulating pipe 105 to be opened and closed, and changes a delivery direction of the airflow. When there is a plurality of perfusion chambers 2, every two adjacent perfusion chambers 2 are communicated with each other through the pressure regulating pipe 105. By connecting all perfusion chambers 2 to one pressure regulating pipe 105, the airflow can be easily adjusted to flow out or in from the perfusion chambers, so as to facilitate the flow of the cell culture fluid, thereby avoiding high shear forces in the perfusion culture system and realizing large-scale cell culture.

If there is only one the perfusion chamber 2, the pressure regulating pipe connected to the perfusion chamber 2 can be used independently. When the scale of the cell culture fluid is large, multiple perfusion chambers 2 can be set side by side and work alternately. Every two adjacent perfusion chambers 2 are connected to each other through the pressure regulating pipe 105, so that air in the perfusion chambers 2 flows out from the perfusion chambers 2 when the cell culture liquid is injected, which facilitates the entry of the cell culture liquid, and air flows into the perfusion chambers 2 when the cell culture liquid is discharged, which increases the pressure in the perfusion chambers and facilitates the discharge of the cell culture fluid.

As shown in FIG. 1 and FIG. 2, the present disclosure provides a perfusion culture system. The perfusion culture system includes a bioreaction module, a perfusion module, and a filtration module.

The bioreaction module includes at least one bioreactor 1.

The perfusion module includes at least one perfusion chamber 2. Each perfusion chamber 2 is connected to a pressure regulating pipe 105 for inflow or outflow of an airflow, and the pressure regulating pipe 105 is provided with a pump that enables the pressure regulating pipe 105 to be opened and closed, and changes a delivery direction of the airflow. When there is a plurality of perfusion chambers 2, every two adjacent perfusion chambers 2 are communicated with each other through the pressure regulating pipe 105.

The filtration module includes at least one filter 3. Each filter 3 has an inlet end and an outlet end opposite to the inlet end, the inlet end is provided with a cell culture fluid inlet, and the outlet end is provided with a permeate outlet and a cell culture fluid outlet.

The at least one bioreactor 1, the perfusion module, and the filtration module are communicated in sequence, and if there are more than two perfusion chambers in the perfusion module, all of the perfusion chambers are set side by side. The cell culture fluid in the at least one bioreactor 1 flows into the at least one perfusion chamber 2 in the perfusion module, and then flows out from the at least one perfusion chamber 2 into the at least one filter 3 for filtration, and a permeate from the at least one filter 3 flows into a harvesting device 5 through the permeate outlet.

In the present disclosure, the at least one bioreactor 1, the at least one perfusion chamber 2, and the at least one filter 3 are communicated through pipelines, and multiple perfusion chambers are set, to achieve large-scale (i.e., 1000-30,000L) perfusion culture. Each perfusion chamber is connected to the pressure regulating pipe, so that the airflow flowed out from the perfusion chambers or flowed in the perfusion chambers can be adjusted, achieving positive pressure control of the perfusion chambers, facilitating the flow of the cell culture fluid, and avoiding high shear forces in the perfusion culture system.

For better recycling of the culture medium, the inlet end of each filter 3 is further provided with a medium inlet, and the medium inlet is connected to a medium supply system 4. The medium supply system 4 is directly connected to the at least one filter 3, and can flush the at least one filter 3 and increase utilization efficiency of the at least one filter 3. The medium supply system 4 includes a medium storage container and a delivery pump 9, and a valve 18 is provided in the pipelines connected to the filters, and the valve 18 is controlled to be opened and closed as needed.

Further, the above medium supply system 4 is connected to the bioreactor 1 through the medium delivery pipe 103, and the medium delivery pipe 103 is provided with a valve 12, and the medium is delivered to the bioreactor 1 under the control of the opening and closing of the valve to better replenish nutrients for the cell culture fluid in the bioreactor 1.

In order to facilitate circulation flow of the cell culture fluid, the outlet of the cell culture fluid of the at least one filter 3 in one embodiment is communicated to the bioreactor 1 through a return pipe 102. As a result, the cell culture fluid filtered by the at least one filter 3 can be returned to the bioreactor 1 through the return pipe 102, which also increases the scale of the cell culture fluid.

In an embodiment, the perfusion module includes only one perfusion chamber 2, as shown in FIG. 1. Atop of the perfusion chamber 2 is communicated with a top of the bioreactor 1 through the pressure regulating pipe 105, and the pressure regulating pipe 105 is provided with a pump 16 that enables the pressure regulating pipe 105 to be opened and closed and changes the delivery direction of the airflow. In an embodiment, the perfusion chamber 2 is communicated with the filter 3 through the pressure regulating pipe 105; when the cell culture fluid in the perfusion chamber 2 is emptied, the valve on the delivery pipe 104 connecting the filter 3 to the perfusion chamber 2 is closed, an on-off valve 11 on an infusion pipe 101 connected to the perfusion chamber 2 is opened, and the pump 16 on the pressure regulating pipe 105 is opened, so that the gas in the perfusion chamber 2 can enter the bioreactor 1, and the cell culture fluid can flow into the perfusion chamber 2. The introduction of the pressure regulating pipe 105 and the pump 16 makes possible positive pressure control of the perfusion chamber 2 (that is, the perfusion chamber 2 is a positive-pressure chamber), which facilitates the circulation of the cell culture fluid and avoids high shear forces in the perfusion culture system.

To better accommodate the large-scale cell culture fluid, the perfusion module in one embodiment has two or more perfusion chambers 2, as shown in FIG. 2. Every two adjacent perfusion chambers 2 are communicated through the pressure regulating pipe 105, and the pressure regulating pipe 105 is provided with a pump that enables the pressure regulating pipe 105 to be opened and closed and changes the delivery direction of the airflow. The pump, in an embodiment, is a pipeline diaphragm pump including valves 17, 8 and 24. The three valves are opened and closed in sequence to pump out the air above the liquid level in one perfusion chamber, thereby enabling the cell culture fluid to flow into the corresponding perfusion chamber, and also enabling air flows out from another perfusion chamber.

The bioreactor 1 is connected to the perfusion chambers 2 through the infusion pipe 101, and the infusion pipe 101 is provided with the on-off valve 11 to enable the infusion pipe 101 to be opened and closed. When there are multiple perfusion chambers, as shown in FIG. 2, on-off valves 11 and 20 are respectively set on branches pipelines of the infusion pipe 101 connected to the perfusion chambers 2, and a normally-open valve 19 is set on the mother pipeline of the infusion pipe 101. To facilitate the delivery of the cell culture fluid to the filtration module from the perfusion chambers 2a, 2b, a delivery end of the perfusion chamber 2a is provided with a valve 23 and a delivery end of the perfusion chamber 2b is provided with a valve 22, so the perfusion chamber 2a is opened or closed by opening or closing the valve 23 and the perfusion chamber 2b is opened or closed by opening or closing the valve 22. When the valves 23, are opened, the cell culture fluid in perfusion chambers 2a, 2b can be discharged. A protection valve 21 and an electromagnetic flow meter 10 are also provided on the delivery pipe 104 connecting the perfusion chambers 2a, 2b to the filtration module, to precisely control the delivery of the cell culture fluid. Besides, each perfusion chamber is provided with one pressure gauge to detect the pressure in the corresponding perfusion chamber, thereby ensuring safety.

Each of the above at least one filter 3 includes hollow-fiber (HF) membranes and each filter 3 can be called an HF filter. HF filters have a long working life and are available in many sizes, constructions, materials, pore sizes and porosities. In addition, the at least one filter 3 is not limited to HF filters, and can also be other isolation devices inserted in hollow fibers.

In an embodiment, the filtration module is provided with two filters 3a, 3b set side by side, and the two filters 3a, 3b work alternately to ensure proper use of the entire system.

In an embodiment, two perfusion chambers and two filters are provided, and the specific working process is as follows: as shown in FIG. 2, the two perfusion chambers 2a and 2b work alternately, the normally-open valve 19 is normally opened, the on-off valve 11 is opened, and the cell culture fluid in the bioreactor 1 is delivered to the perfusion chamber 2a through the infusion pipe 101; each perfusion chamber 2 is provided with a liquid level meter, and the liquid level meter is used to detect whether the cell culture fluid in each perfusion chamber reaches the maximum level or the minimum level to control the cell culture fluid to flow out from or to flow into the corresponding perfusion chamber. When the cell culture fluid in the perfusion chamber 2a reaches the maximum working volume, the on-off valve 11 is closed, and the on-off valve 20, the valve 23 and the valve 14 are opened. At this time, the cell culture fluid in the bioreactor 1 is delivered to the perfusion chamber 2b through the infusion pipe 101, and the valve 24, the valve 8, and the valve 17 in the pump are opened in sequence, to pump the air above the liquid level in the perfusion chamber 2b into the perfusion chamber 2a. That is, the flow direction of the airflow in the pressure regulating pipe 105 is from left to right (with reference to FIG. 2), and the compressed air is delivered into the perfusion chamber 2a under the control of the mass flow controller 6 at the same time, so that the perfusion chamber 2a has sufficient perfusion pressure to enable the cell culture fluid in the perfusion chamber 2a to flow into the filters 3 through the delivery pipe 104. The two filters 3a, 3b work alternately to ensure that one of the two filters is always in a working state; a harvesting pump 7 delivers the permeate from the filters 3a, 3b to the harvesting device 5 through the permeate outlet, and the cell culture fluid is returned to the bioreactor 1 through the return pipe 102.

When the cell culture fluid in the perfusion chamber 2b reaches the maximum volume and the cell culture fluid in the perfusion chamber 2a reaches the minimum level, the on-off valve 11 is opened, the on-off valve 20 is closed, the valve 22 is opened, the valve 23 is closed, and then the cell culture fluid in the bioreactor 1 is delivered to the perfusion chamber 2a through the infusion pipe 101. At this time, the valve 17, the valve 8, and the valve 24 in the pump are opened in sequence, to pump the air above the liquid level in the perfusion chamber 2a into the perfusion chamber 2b. That is, the flow direction of the airflow in the pressure regulating pipe 105 is from right to left (with reference to FIG. 2), and the compressed air is delivered into the perfusion chamber 2b under the control of the mass flow controller 6 at the same time, so that the perfusion chamber 2b has sufficient perfusion pressure to enable the cell culture fluid in the perfusion chamber 2b to flow into the filters 3 through the delivery pipe 104.

The perfusion chambers 2a, 2b are used sequentially and repeatedly to ensure the normal delivery of the cell culture fluid and improve the scale of the whole system.

On one hand, the medium supply system 4 flushes the filters 3, on the other hand, the medium supply system 4 is connected to the bioreactor 1 through the medium delivery pipe 103 to replenish the medium for the bioreactor 1.

In order to make the perfusion chambers better deliver the cell culture fluid to the filters, in one embodiment, each perfusion chamber 2a or 2b is connected to the mass flow controller 6 through an airflow delivery pipe. The airflow delivery pipe is provided with a gas filter 15 or/and a switching valve 14. The gas filter 15 guarantees the sterility of air supplied to each perfusion chamber under the control of the mass flow controller 6.

The mass flow controller 6 and other actuators such as valves or pumps are controlled by action commands sent by a controller, which may be configured according to actual process parameters. In one embodiment, the mass flow controller 6 is a conventional mass flow controller and can be used according to methods known to those skilled in the art.

The present disclosure also provides an embodiment where there are three and more perfusion chambers in the perfusion culture system, in which case every two adjacent perfusion chambers can be connected through one pressure regulating pipe, or the perfusion chambers are connected to the bioreactor through the pressure regulating pipe to realize positive pressure control of the perfusion chambers. In this embodiment, the multiple perfusion chambers 2 work alternately and/or work simultaneously to achieve the flow of the large-volume ell culture fluid and facilitate permeate harvesting, so that the whole system can be easily controlled for implementation. The materials of the perfusion chambers may include 316L stainless steel and heat-resistant borosilicate glass.

As described above, the perfusion culture system of the present disclosure achieves large-scale (i.e., 1000-30,000 L) perfusion culture, and avoids high shear forces in the perfusion culture system by introducing positive-pressure perfusion chambers . At the same time, the perfusion culture system allows for online sterilization and easy operation.

The above embodiments are illustrative of the principles and benefits of the disclosure rather than restrictive of the scope of the disclosure. Persons skilled in the art can make modifications and changes to the embodiments without departing from the spirit and scope of the disclosure. Therefore, all equivalent modifications and changes made by persons skilled in the art without departing from the spirit and technical concepts disclosed in the disclosure shall still be deemed falling within the scope of the claims of the disclosure.

What is claimed is:

1. A perfusion module, wherein the perfusion module comprises at least one perfusion chamber, wherein each perfusion chamber is connected to a pressure regulating pipe for inflow or outflow of an airflow, and the pressure regulating pipe is provided with a pump that enables the pressure regulating pipe to be opened and closed and changes a delivery direction of the airflow; wherein when there are a plurality of perfusion chambers, every two adjacent perfusion chambers are communicated with each other through the pressure regulating pipe.

2. A perfusion culture system, comprising:
a bioreaction module, comprising at least one bioreactor;
a perfusion module, comprising at least one perfusion chamber, wherein each perfusion chamber is connected to a pressure regulating pipe for inflow or outflow of an airflow, and the pressure regulating pipe is provided with a pump that enables the pressure regulating pipe to be opened and closed and changes a delivery direction of the airflow; wherein when there are a plurality of perfusion chambers, every two adjacent perfusion chambers are connected to each other through the pressure regulating pipe;
a filtration module, comprising at least one filter, wherein each filter has an inlet end and an outlet end opposite to the inlet end, the inlet end is provided with a cell culture fluid inlet, and the outlet end is provided with a permeate outlet and a cell culture fluid outlet;
wherein the at least one bioreactor, the perfusion module, and the filtration module are communicated in sequence, and if there are more than two perfusion chambers in the perfusion module, all of the perfusion chambers are set side by side; wherein a cell culture fluid in the at least one bioreactor flows into the at least one perfusion chamber in the perfusion module, and then flows out from the at least one perfusion chamber into the at least one filter for filtration, and a permeate from the at least one filter flows into a harvesting device through the permeate outlet.

3. The perfusion culture system according to claim 2, wherein the inlet end of each filter is further provided with a medium inlet, and the medium inlet is connected to a medium supply system.

4. The perfusion culture system according to claim 2, wherein the cell culture fluid outlet of each filter is connected to the at least one bioreactor through a return pipe, and the cell culture fluid filtered by each filter is returned to the at least one bioreactor through the return pipe.

5. The perfusion culture system according to claim 2, wherein the medium supply system is connected to the at least one bioreactor through a medium delivery pipe.

6. The perfusion culture system according to claim 2, wherein when the perfusion module comprises only one perfusion chamber, a top of the perfusion chamber is communicated with a top of the at least one bioreactor through the pressure regulating pipe.

7. The perfusion culture system according to claim 2, wherein the pump is a diaphragm pump.

8. The perfusion culture system according to claim 2, wherein each perfusion chamber is connected to an airflow mass flow controller through an airflow delivery pipe.

9. The perfusion culture system according to claim 8, wherein the airflow delivery pipe is provided with a gas filter or/and a switching valve.

10. The perfusion culture system according to claim 8, wherein the at least one bioreactor is connected to the at least one perfusion chamber through an infusion pipe, and the infusion pipe is provided with an on-off valve to enable the infusion pipe to be opened and closed.

11. The perfusion culture system according to claim 2, wherein the filtration module is provided with two filters set side by side, and the two filters work alternately.
